(19) United States
(12) Patent Application Publication
(10) Pub. No.: US 2002/0077286 A1
(43) Pub. Date: Jun. 20, 2002

(54) Mechanically activated channel blocker

(76) Inventors: Frederick Sachs, Eden, NY (US); Janice H. Johnson, Salt Lake City, UT (US); Thomas Suchyna, Amherst, NY (US)

Correspondence Address: Ranjana Kadle, Hodgson Russ LLP, Suite 2000, One M&T Plaza, Buffalo, NY, 14203-2391, US

(21) Appl. No.: 09/827,814

(22) Filed: Apr. 6, 2001

Related U.S. Application Data
(60) Provisional application No. 60/195,528, filed on Apr. 7, 2000.
(60) Provisional application No. 60/277,071, filed on Mar. 19, 2001.

(51) Int. Cl. A61K 38/17; C07K 14/435

(52) U.S. Cl. 514/12; 530/350

(57) ABSTRACT

The present invention discloses a 35 amino acid peptide (SEQ ID NO: 1) that blocks stretch-activated ion channels. The peptide, designated as GsMTx-4, is present in the venom of the spider Grammostola spatulata. The present invention also discloses a method of purifying the peptide GsMTx-4 from the spider venom and a method for inhibition of stretch activated ion channels in a cell. This peptide can be used for the treatment of cardiac arrhythmias.

This application claims priority to U.S. Provisional application Ser. No. 60/195,528, filed on Apr. 7, 2000 and to U.S. Provisional application Ser. No. 60/277,071 filed on Mar. 19, 2001, the disclosures of which are incorporated herein by reference.

### FIELD OF THE INVENTION

The present invention relates to the field of stretch-activated channels. More particularly the present invention provides a peptide that blocks stretch-activated channels, such as those associated with cardiac arrhythmias.

### DISCUSSION OF RELATED ART

Cardiac fibrillation is a frequent cause of sudden death. Atrial fibrillation is the most common sustained cardiac arrhythmia to occur in humans, second only to valve disease, hypertension, or heart failure. Atrial fibrillation is often associated with passive stretching of the arterial chamber arising from haemodynamic or mechanical dysfunction of the heart. It has been suggested that abnormal mechanical factors induce electrophysical changes conducive to arrhythmia via “mechanoelectric feedback”. Stretch-activated channels (SACs) have been postulated as a mechanism of mechanoelectric feedback and they may play a role in the genesis of cardiac arrhythymias.

Mechanosensitive ion channels (MSCs), of which SACs are an example, were discovered in tissue cultured skeletal muscle cells using single channel patch clamp recordings. They have since been found in both the plant and animal kingdoms and in the cells of most tissues, including myocardial tissue. Most of them open with increasing membrane tension (stretch-activated channels (SACs)), but a few are tonically active and close with increasing tension (stretch-inactivated channels (SICs)). It is now well recognized that myocardial stretch can cause arrhythmias due to stretch-induced depolarizations.

Ion selectivity of the MSC channel family is variable, as in the case of voltage-activated or ligand-activated channel families. In the animal cells, the most common forms are cation selective and, more particularly, potassium selective. The cation channels will pass divalents such as Ca⁺² and Ba⁺² as well as monovalents. Due to their ability to pass Ca⁺², effects of cationic MSCs are potentially complicated. Even under voltage clamp conditions, incoming Ca⁺² may activate other channels, such as Ca²⁺ activated Cl⁻ channels, a link that has been invoked in the regulation of cell volume.

SACs have been implicated as either activators or modifiers of many different cellular responses to mechanical stimuli including modification of electrical and contractile activity of muscle tissue. Consequently, SACs have been implicated in mechanical sensitivity of the heart. Mechanical stimulation of cardiac myocytes and whole heart preparations can cause depolarization, extrasystoles and arrhythmias (Hu et al., 1997; J Mol Cell Cardiol 29:1511-1523). Also, chronic hemodynamic stress that leads to congestive heart failure (CHF) and the accompanying cellular hypertrophy may be initiated by stretch- or swelling-activated currents (Sachs, 1988; Crit Rev Biomed Eng 16:141-169; Vandenberg et al., 1996; Cardiovasc Res 32:85-97; Clemo et al., 1997; J Gen Physiol 110:297-312).

SACs are the only major class of ion channels for which a specific inhibitor is not known. Gd³⁺ is the best known blocker of SACs (K_{D}'s ranging from 1-100 mM) and is widely used to identify these channels. However, Gd³⁺ also blocks a variety of other channels such as L- and T-type Ca²⁺ (Biagi et al., 1990, Am. J. Physiol., 264:C1037-1044), K⁺ delayed rectifier, voltage-gated Na⁺ (Elinder et al., 1994, Biophys. J., 67:71-83) and Ca²⁺ ER release channels (Kluesener et al., 1995, EMBO J., 14:2708-2714). A variety of blockers for voltage- and ligand-gated channels (e.g. amiloride, cationic antiobiotics, tetrodotoxin, tetraethylammonium, quinidine, diltiazem and verapamil) exhibit low affinity blocking activity against SACs (for review see Hamill et al., 1996, Pharmacol Rev 48:231-252; Sachs et al., 1998; M. P. Blaustein, R. Greger, H. Grunicke, r. Jahn, L. M. Mendell, A. Miyajima, D. Pette, G. Schultz, and M. Schwieger, editors; Springer, Berlin 1-78).

Thus, while several studies point to a role for SACs in mechanical sensitivity, a lack of specific SAC agent has hampered the development of SAC based approach to the treatment of arrhythmias. Consequently, there is an ongoing need to identify agents that can block SACs. Such agents could be useful in influencing events associated with cardiac arrhythmias, and could be the first of a new class of anitarrhythmic agents to be directed against the causes rather than the symptoms of fibrillation.

### SUMMARY OF THE INVENTION

The present invention discloses a 35 amino acid peptide (SEQ ID NO: 1) that blocks stretch-activated ion channels. The peptide, designated as GsMTx-4, is present in the venom of the spider Grammostola spatulata.

The present invention also discloses a method of purifying the peptide GsMTx-4 from the spider venom. The method comprises the steps of multiple fractionations of the spider venom.

The present invention also provides a method for inhibition of stretch activated ion channels in a cell. The method comprises the step of applying to the cell a sufficient amount of the peptide GsMTx-4.

The present invention also provides a method for the treatment of cardiac arrhythmias. The method comprises the step of applying the peptide GsMTx-4 to a heart tissue that is exhibiting arrhythmia.

The present invention also discloses a method for identifying the presence of the peptide GsMTx-4 in venom. The method comprises the steps of fractionating the venom sample, and assaying the effect of the fractions on SAC activity using patch clamp recordings.

### BRIEF DESCRIPTION OF THE DRAWINGS

FIG. 1A is a chromatogram of Grammostola whole venom produced by a 40 minute linear gradient from 15-55% acetonitrile at a flow rate of 3.5 ml/minute. Pool fractions are labeled at the bottom. The percent acetonitrile is indicated by the dotted line overlaying the chromatogram.

FIG. 1B is a chromatogram of fraction 9 from FIG. 1A.

FIG. 1C is a chromatogram of fraction B from FIG. 1B.

FIG. 1D is a chromatogram of the single peak from FIG. 1C.

FIG. 2 is a representation of the sequence of GsMTx-4 showing homology to other ion channel peptide toxins. Cysteine motif residues are included in boxes. Dark shaded residues in the comparison peptide sequences are identical to GsMTx-4, while lighter shaded residues are similar.

FIGS. 3A-3D are representations of cell-attached and outside-out patches from adult activated astrocytes showing stretch sensitive channels with similar unitary conductance profiles but different gating properties. FIG. 3A is a representation of cell attached patch from adult astrocytes showing single channel recording above average patch currents. FIG. 3B is a representation of single channel recording for an outside out patch showing the presence of two to three channels. FIG. 3C is a representation of a unitary current-voltage plot fitted with a second-order polynomial showing inward rectification for channels in cell-attached patches (n=11). FIG. 3D is a representation of a unitary current voltage plot fitted with a second-order polynomial showing inward rectification for channels in outside-out patches(n=16).

FIG. 4 is a representation of blockage of SACs by GsMTx-4 in outside-out patches.

FIGS. 5A-5D are representations of rate of blocking by GsMTx-4 as determined by superfusion of activated SACs in outside-out patches. FIG. 5A is a control trace (top) and current record in the presence of GsMTx-4 (bottom). The control trace was generated from 37 pressure steps applied to seven different patches held at −50 mV, with pressure levels ranging from 35-70 mm Hg. FIG. 5B is a superimposition of the average current records from 5A. FIG. 5C is the result of subtracting the control current race from the GsMTx-4 trace. FIG. 5D is the current trace during GsMTx-4 application fitted with a single exponential yielding a time constant of 594±10 ms. The fit is shown displaced from the data for clarity.

FIGS. 6A is a representation of the dissociation rate of GsMTx-4, determined from the recovery rate of SAC current on washout. SAC currents were activated by 500-ms pressure steps at 2-s intervals in outside-out patches held at −50 mV.

FIG. 6B shows the recovery kinetics fitted to a single exponential with a time constant of 4.7±1.7 s.

FIGS. 7A-G are representations of the effect of GsMTx-4 on whole cell swelling-activated current measured in astrocytes exposed to hypotonic saline. FIG. 7A shows resting whole-cell currents in isotonic saline produced by the waveform shown in 7E. (isotonic saline is normal bath saline with 80 mM NaCl replaced by 160 mM mannitol). Current scale bar is shown to the right. Swelling-activated currents were recorded after the cell had been exposed for 30 s to hypotonic saline. FIG. 7B is with isotonic saline minus 140 mM mannitol. FIG. 7C shows perfusion of hypotonic saline with 5 μM GsMTx-4. FIG. 7D shows swelling currents partially recovered about 4 minutes after washout of GMsTx-4. FIG. 7F shows peak swelling activated currents at 100 mV from two different cells (a and b) decreased over successive exposures to hypotonic solution. FIG. 7G shows an I-V plot of the average swelling-activated peak currents from six cells measured 30-40 s after hypotonic exposure.

FIG. 8 is a representation of the effect of DIDS on swelling-activated currents in adult astrocytes.

FIGS. 9A-D are representations of ionic currents (A-C) and cell volumes (D) measured during perforated patch voltage clamp (E_{hold}=−80 mV) of ventricular myocytes from rabbits with aortic regurgitation-induced CHF. FIG. 9A shows osmotic shrinkage in the control solution reduced both inward and outward currents. FIG. 9B shows that toxin reduced the inward currents in 1.0 T, but the currents in 1.5 T were unaffected. FIG. 9C shows shrinkage sensitive current due to inhibition of cationic SACs and anionic swelling currents. FIG. 9D show that the toxin did not affect membrane currents when SACs were inhibited by osmotic shrinkage.

FIG. 10A is a representation of bipolar electrograms showing an incerase in atrial fibrillation (AF) with pressure, becoming sustained at 12.5 cm H₂O.

FIG. 10B is a representation of the induction of AF lasting more than 2 seconds for control (∘) and in the presence of 170 nM GsMTx-4 (●). Dashed line indicates the response after 20-min washout.

FIG. 10C shows the duration of AF (n=7) as a function of pressure (mean ± standard error). GsMtx-4 (170 nM) decreased the average time to spontaneous recovery from AF (asterisks, P less than 0.05).

FIG. 10D shows that GsMtx-4 did not block stretch-induced shortening of the refractory period (n=10).

### DETAILED DESCRIPTION OF THE INVENTION

The present invention discloses the identification, isolation and sequencing of a novel peptide present in the venom of Grammostola spatulata. The peptide is 35 amino acid in length (SEQ ID NO: 1). This peptide is designated herein as GsMTx-4. This peptide contains six cysteine residues and does not show significant homology to any other peptide toxin.

The present invention also discloses a method for purifying the peptide from spider venom and a method for using the peptide for blocking SACs. GsMTx-4 is useful for treatment of cardiac arrhythmias in mammals. It can be prepared by purification of the Grammostola spatulata venom. The venom is commercially available (Spider Pharm, Feasterville, Pa.) or may be elicited from the spider by standard techniques such as electrical stimulation.

GsMTx-4 can be isolated from spider venom by serial fractionation using standard chromatographic techniques. In a preferred embodiment, fractionation of the spider venom is carried out using reverse phase high performance liquid chromatography (HPLC). Reverse phase HPLC can be performed using C-8 or C-18 silica columns and trifluoroacetic acid/acetonitrile buffer system. C-8 and C-18 silica columns are commercially available (Mac-Mod Analytical, Inc., West Chester, Pa.).

The peptide GsMTx-4 can also be prepared by chemical synthesis using automated or manual solid phase methods. Such technologies are well known in the art. For example, such technologies are described in E. Atherton and R. C. Sheppard, Solid Phase Peptide Synthesis: A Practical Approach, IRL Press/Oxford Univeristy Press, Oxford, England, 1989; and M. Bodanzky, Peptide Chemistry: A Practical Textbook, Springer-Verlag, New York, N.Y., 1988. Thus, the peptide GsMTx-4 can be synthesized using Fmoc chemistry or an automated synthesizer. Depending upon quantitative yields, production of the linear reduced peptide can be performed in either a single process or in two different processes followed by a condensation reaction to join the fragments. A variety of protecting groups can be incorporated into the synthesis of linear peptide so as to facilitate isolation, purification and/or yield of the desired peptide. Protection of cysteine residues in the peptide can be accomplished using protective agents such as triphenylmethyl, acetamidomethyl and/or 4-methoxybenzyl group in any combination.

Further, the peptide GsMTx-4 may also be prepared by recombinant DNA technology. A DNA sequence coding for the peptide is prepared, inserted into an expression vector and expressed in an appropriate host cell. The expressed peptide can then be purified from the host cells and/or culture medium. Methods for preparing DNA coding for the peptide and expression of DNA are well known to those skilled in the art and are found for example, in Sambrook et al., (1989) Moelcular Cloning: A Laboratory Manual, Cold Spring Harbor, N.Y., S. L. Berger and A. R. Kimmel, Eds., Guide to Molecular Cloning Techniques: Methods in Enzymology, vol 152, Academic Press, San Diego, Calif., 1987, and in E. J. Murray, Ed., Gene Transfer and Expression Protocols: Methods in Molecular Biology, vol 7, Humana Press, Clifton, N.J., 1991.

The peptide GsMTx-4 of the present invention can be prepared for pharmaceutical use by incorporation with a pharmaceutically acceptable carrier or diluent. The peptide can be formulated into tablets, capsules, caplets and the like. Suitable carriers for tablets include calcium carbonate, starch, lactose, talc, magnesium stearate and gum acacia. The peptide can also be formulated for oral, parenteral or intravenous administration in aqueous solutions, aqueous alcohol, glycol or oil solutions or emulsions. The peptide can also be formulated for inhaling by encapsulating to facilitate alveolar absorption as has been done for insulin (Inhale Theraputic Systems, Zan Carlos, Calif., www. Inhale.com). Pharmaceutical compositions suitable for such routes of administration are well known in the art. For example, suitable forms and compositions of pharmaceutical preparations can be found in Remington's Pharmaceutical Science, 1980, 15^{th} ed. Mack Publishing Co., Easton, Pa. Thus, the peptide GsMTx-4 can be administered orally, parentarally, intravenously, intramuscularly or intranasally.

The amount of GsMTx-4 in the pharmaceutical composition can be determined by empirical methods. Those skilled in the art will recognize that the dosage administered to a particular individual will depend on a number of factors such as the route of administration, the duration of treatment, the size and physical condition of the individual, and the patient's response to the peptide including the side effects. Antiarrhythmic concentrations of another peptide toxin from spider venom are disclosed in U.S. Pat. No. 5,756,663.

As used herein the antiarrhythmic activity refers to the activity of the peptide GsMTx-4 in blocking stretch-activated channels in myocytes.

The following examples describe the various embodiments of this invention. These examples are illustrative and are not intended to be restrictive.

### EXAMPLE 1

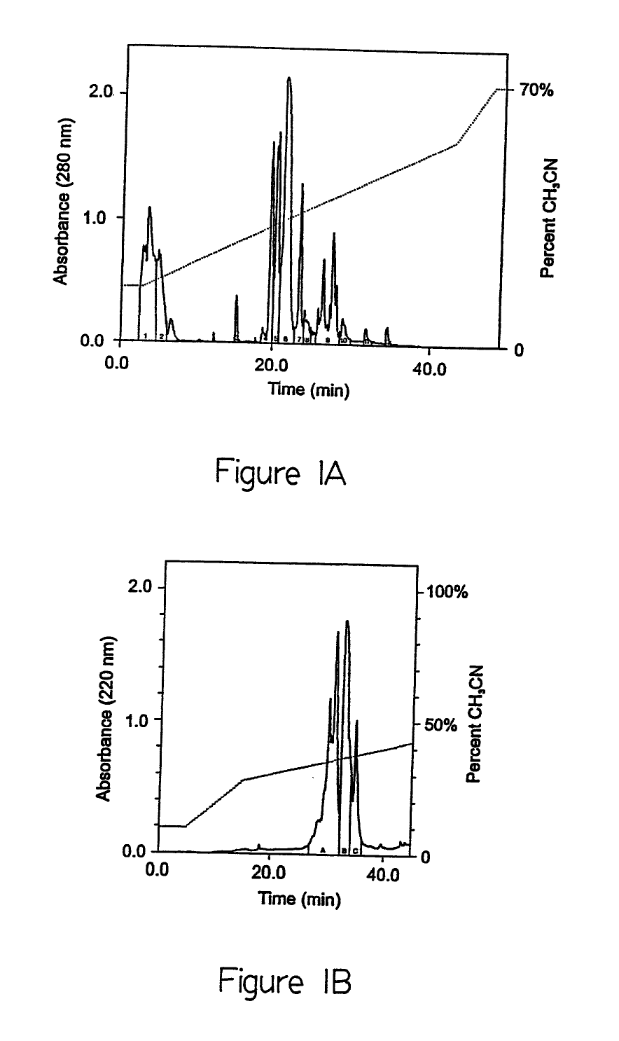

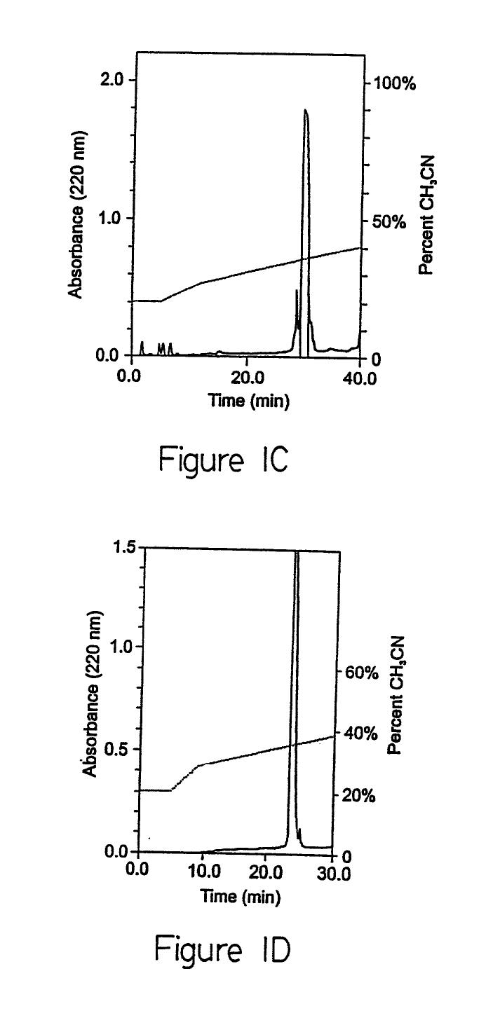

This embodiment describes the isolation and molecular characterization of the peptide. For the isolation of the peptide, Grammostola spatulata (Theraphosidae) spiders were obtained from a captive population at Hogel Zoo (Salt Lake City, Utah). The arachnid species Grammostola spatulata, commonly referred to as the Chilean pink tarantula spider, is a member of the Theraphosidae family and the Chelicerata order. The Grammastola species have recently been reassigned to the genus Phixotricus , but Grammastola is used here to maintain consistency with prior biomedical publications. Venom was produced by an electrical milking procedure (Bascur et al., 1982, Toxicon, 20:795-796) and stored at −80 C. The venom was fractionated by high-performance liquid chromatography, incorporating Beckman System Gold 126 solvent delivery and 168 photodiode-array detector modules (Beckman Instruments, Fullerton, Calif.) using linear gradients with a flow rate of 3.5 ml/min unless noted. Whole venom (825 μl) was separated into eleven 75 μl aliquots that were each diluted to 2 ml each with 15% B. Solvent A was 0.1% TFA in water and solvent B was 0.1% TFA in acetonitrile. The diluted venom was fractionated on a (Zorbax RX-C8, 9.4×250 mm, 5 μm, 300 A; Mac-Mod Analytical, Inc., Chadds Ford, Pa.) reversed-phase (RP) column equilibrated in 15% B. The column was developed with a 40 min gradient (15-55% B) begun 3 min after injection of the sample with a flow of 3.5 ml/min. The effluent was monitored at 280 nm and fractions collected as noted on the chromatogram (FIG. 1A). Similar fractions from all eleven chromatographies were combined, lyophilized and tested for bioactivity. The assay samples were dissolved in 140 mM NaCl, 10 mM Hepes, 5 mM KCl, 2 mM MgSO4 to a final dilution of 1:1000, relative to whole venom, for testing on outside-out patches. Several of the pools showed partial block of the SACs (as described below), but only pool 9 gave consistent, complete block of the channels.

Further purification of pool 9 (FIG. 1B) was achieved by RP chromatography on a Vydac C18 column (10×250 mm, 5 μm, 300 A; The Separations Group, Hesperia, Calif.) equilibrated in 10% B. Lyophilized pool 9 was dissolved in 4 ml of 10% B and chromatographed in 1 ml portions eluting with a 10 min gradient (10-28% B), followed by a 64 min gradient (28-60% B). The first gradient was begun 5 min after injection of the sample, the effluent was monitored at 220 nm and three fractions were collected. Corresponding fractions from the four chromatographies were combined, lyophilized and assayed as described above. Only pool B showed block of the SACs.

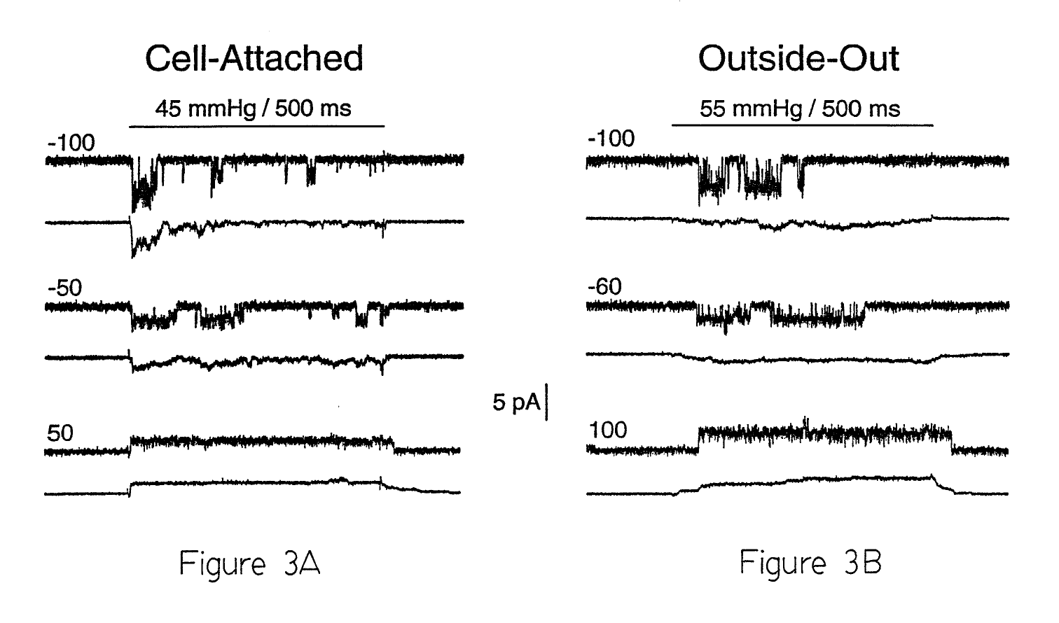

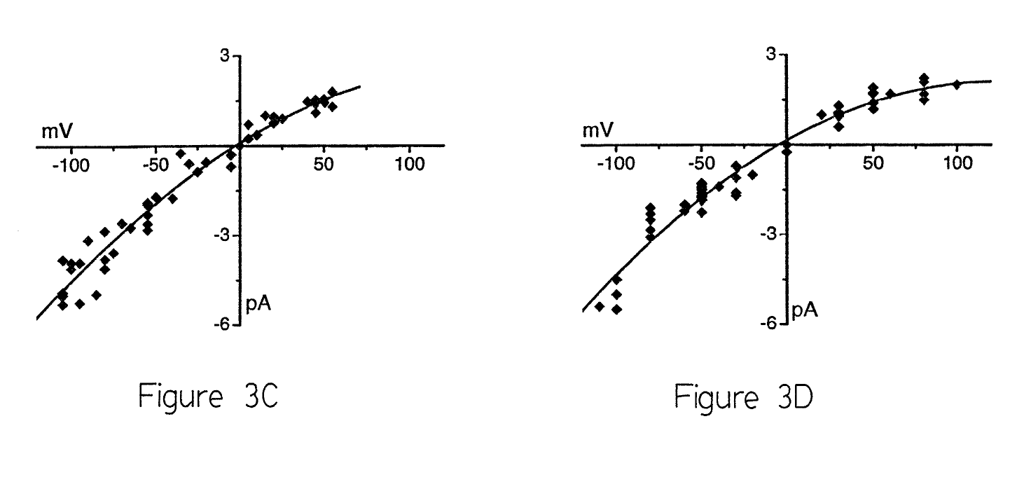

Therefore, pool B was subjected to a final RP chromatography to remove a small amount of earlier and later eluting peptides. Pool B was diluted to 4 ml with 20% solvent B and 0.5 ml portions chromatographed on the Zorbax column described above, eluting with a 7 min gradient (20-27% B), followed by a 46 min gradient (27-50% B) and the effluent was monitored at 220 nm (FIG. 3C). The first gradient was begun 5 min after injection of the sample. The active peptide, GsMTx-4 eluted between 29.5 and 30.5 min. Corresponding fractions from the eight chromatographies were pooled to give 7.5 mg of GsMTx-4. The average yield of GsMtx-4 from several purifications was 8 mg/ml of venom fractionated, which corresponds to a concentration of ^{˜}2 mM in whole venom. The purity of the final product used in single channel and whole cell assays was assessed by analytical chromatography (Aquapore RP300 C8 column, 4.6×150 mm, 5 um, 300 Å; PE Biosystems, Forest City, Calif.) eluting with a 40 min linear gradient (15-55% B) with a flow of 1 ml/min monitored at 220 nm (FIG. 1D). Elution with a gradient of methanol/water (0.1% in TFA) gave a similar profile with a longer retention time, but revealed no other impurities.

The peptide was further purified by microbore reverse phase-HPLC (0.8 mm×250 mm C18 column, with a linear gradient from 0.1% TFA-15% CH₃CN to 0.1% TFA-70% CH₃CN in 90 min, flow rate 40 μl/min, monitored at 214 nm). The toxin peak was collected at 24.6 min. The HPLC fraction (^{˜}1 nmol) was dried down and taken up in 80 μl 8 M guanidine HCL-100 mM TRIS-5 mM tributylphosphine at pH 8.5 and incubated for 8 h at 55° C. N-isopropyliodoactamide (1 mg in 20 μl MeOH+80 μl TRIS) was added and the solution was incubated for an additional 2 h at room temperature. The reduced and alkylated peptide was then desalted by HPLC on a C18 column as described above (elution time 30.1 min). N-terminal sequencing was carried out on an ABI 477 after loading the reduced and alkylated peptide on PVDF membrane.

Digestion with BNPS-skatole (Fontana, 1972) was carried out by dissolving the purified reduced and alkylated peptide in 50 ul 0.1% TFA and 15 μl BNPS-skatole. The solution was incubated at room temperature for 8 h. The digestion products were separated by HPLC as described above. Two main peaks were collected and sequenced by Edman degradation. Asp-N digestion (Wilson 1989) was performed by dissolving the purified reduced and alkylated peptide in 100 mM TRIS, pH 8.0 and treating with 1% (w/w) Asp-N for 20 h at 35 C. The fragments were separated and analyzed by mass spectrometry prior to Edman degradation.

For determination of the molecular weight by mass spectrometry, one micro-liter of the sample solution (intact toxin or fragments) in 0.1% TFA (or the HPLC elution solvent) was mixed on the sample plate with 1.0 μl of a saturated solution of 4 hydroxy-α-cyanocinnamic acid in 1:1 CH₃CN:0.1% aq.TFA. The solution was air dried before introduction into the mass spectrometer. Spectra were acquired on a PerSeptive Biosystems Voyager Elite MALDI-TOF (matrix assisted laser desorption ionization-time of flight) instrument operated in linear reflectron delayed extraction mode (50-100 nsec). The instrument was equipped with a nitrogen laser (3 nsec pulse). The acceleration potential was 22 kV.

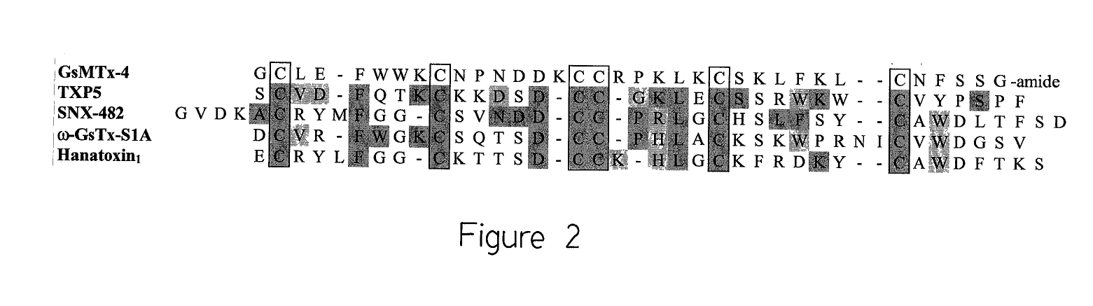

MALDI-MS analysis showed the molecular weight of the native toxin was 4093.90 (MH+ion). The alkylated and reduced toxin displayed a peak at m/z 4690, indicating 3 disulfide bonds or six cysteine residues were present. N-terminal sequencing was followed by sequencing of two different C-terminal fragments produced by enzymatic digests with BNPS-skatole and Asp-N. The predicted mass up to, but not including the C-terminal amino acid, is 4019.85 if the protons for 3 disulfide bonds are subtracted. The difference between the measured unprotonated mass of 4092.9 and the latter (4019.85) is 73.05, which is the mass of a glycineamide. The mass accuracy of the MALDI-MS analysis is approximately ±0.1 D with internal calibration. The final sequence shown in FIG. 2 (also SEQ ID NO: 1) is 35 amino acids in length with the C-term glycine amide added.

The six cysteine residues included in boxes form an ICK motif (CX₃₋₇CX₃₋₆CX₀₋₅CX₁₋₄CX₄₋₁₃C) commonly observed in many other peptide toxins from both terrestrial and aquatic animal venoms (Narasimhan et al., 1994, Nature Structural Biol., 1:850-852; Norton et al., 1998, Toxicon, 36:1573-1583). GsMTx-4 shows less than 50% homology to any other peptide toxin. Other tarantula toxins which block voltage gated Ca²⁺ and K⁺ channels show the highest percentage of similarity to GsMTx-4 as illustrated by the amino acid alignment in FIG. 2. A K⁺ channel toxin labeled protein 5 from Brachypelma smithii (Kaiser et al., 1994, Toxicon, 32:1083-1093; Norton et al., 1998, supra) shows ^{˜}50% total sequence similarity. The most significant regions of homology occur within the cysteine motif. Besides the conserved cysteine motif, there are 3 other residues (F4, D13 and L20) that are conserved in all five toxins. Like the positively charged -conotoxin and -agatoxin families of Ca²⁺ channel blockers, GsMTx-4 carries an overall positive charge (+5).

### EXAMPLE 2

This embodiment describes the effect of the peptide GsMTx-4 on SACs. To illustrate this, adult rat astrocytes which are known to have SACs, were used. Adult rat astrocytes were cultured according to standard methods. Briefly, activated adult astrocytes isolated from gelatin-sponge implants from adult Sprague-Dawley rat brains obtained according to the method of Langan et al. (1995, Glia, 14:174-184) were used at passage numbers 2-4. Astrocytes were maintained in DMEM, 10% fetal bovine serum and 1% Penicillin/Streptomycin and were used in experiments between 2 and 5 days after passage. Cells between passage 4 and 35 expressed SACs with the same properties. Both stellate process bearing cells and flat polygonal (fibroblast-like) cells were used.

The cultured astrocytes were used for single channel and whole cell recorndings. For single channel patch clamp, patch voltage was controlled by an Axopatch 200B (Axon Instruments, CA) and currents were recorded directly onto computer disk via a Labmaster DMA Ver. B (Scientific Instruments, CA) board controlled by pClamp6-Clampex acquisition software (Axon Instruments, CA). Currents were sampled at 10 KHz and low-pass filtered at 2 KHz through the 4 pole Bessel filter on the Axopatch 200B. Experimental voltage protocols were controlled by pClamp6-Clampex. All potentials are defined with respect to the extracellular surface.

Electrodes were pulled on a Model PC-84 pipette puller (Brown-Flaming Instruments, CA), painted with Sylgard 184 (Dow Corning Corp. Midland, Mich.) and fire polished. Electrodes were filled with KCl saline (KCl 140 mM, EGTA 5 mM, MgSO₄ 2 mM, Hepes 10 mM, pH 7.3) and had resistances ranging from 3-8 MW. Bath saline consisted of NaCl 140 mM, KCl 5 mM, MgSO₄ 1 mM, CaCl₂ 1 mM, glucose 6 mM and Hepes 10 mM, pH 7.3.

Pressure and suction were applied to the pipette by a pressure clamp. Pressure values represent pressure in the pipette, i.e., the intracellular side of the membrane in outside-out patches. Suction applied to a cell-attached patch has the same sign as pressure applied to an outside-out patch. The rise time of pressure changes at the tip were determined by monitoring the rate of current change when pressure steps were applied to an electrode containing 150 mM KCl solution and placed in a water bath. The t₁₀₋₉₀ was ^{˜}5 ms as determined by exponential fits to the current decay. Perfusion of toxin samples was performed by a pressurized bath perfusion system BPS-8 (ALA scientific instruments, NY) with 8 separate channels.

Off-line data analysis was performed with pClamp6 analysis software and Origin 5.0. Maximal unitary channel currents were determined via Guassian fits to the peaks of all points amplitude histograms generated from records containing 1-3 channels. Many current records displayed more than 3 channel openings (maximal single channel currents plus subconductance states) and were impossible to fit using Pstat software. Some of these records were analyzed by determining all of the step-like changes in current during the pressure application and selecting the average maximal current level as the unitary current. The data analyzed by this method was in good agreement with the unitary current levels determined by analysis of all points amplitude histograms from single channel patches.

The presence of stretch-activated channels in the astrocytes is shown in FIGS. 3(A-D). Representative single channel current recordings are shown above average patch currents from a cell-attached patch (A) containing a single channel, and an outside out patch (B) containing 2-3 channels. Cell-attached patch recordings were made with 140 mM KCl pipette saline, and outside-out patch recordings are with symmetrical 140 mM KCl pipette solutions. Pressure steps (indicated by the bar at the top) were applied to the patches at different holding potentials shown to the left of each recording. Voltages are relative to the extracellular side. Average current records were calculated from multiple pressure steps (ranging from 5-15 steps) at each voltage. In cell-attached mode, channel adaptation, lower P_{O} and multiple sub-conductance states are apparent at negative potentials. Channels in outside-out patches from astrocytes show slow voltage dependent activation and lower P_{O} at negative potentials. Unitary current-voltage plots were fitted with a second order polynomial and show inward rectification for channels in cell-attached (C, n=11 patches) and outside-out (D, n=16 patches) patches. Voltages for cell-attached data points were corrected for the average resting membrane potential measured in the whole cell configuration. Each point represents an average current calculated from applying multiple pressure steps to a single patch.

These data indicate that in cell-attached patches, activated adult astrocytes express primarily one type of SAC that can be activated by both pressure and suction (FIG. 3A, only pressure data shown). Observation from more than 100 patches typically showed 2-5 channels/patch. SAC activity in cell-attached patches was sensitive to the level of suction used in seal formation. Channels were rarely observed when >10 mmHg of suction was used during seal formation, whereas >90% of patches showed channel activity with <10 mmHg. With 140 mM KCl in the electrode, the single channel conductance inwardly rectified being 46 pS at −100 mV, but only 21 pS at +100 mV (FIG. 3C). Channel activity was normally initiated by applying between 25-35 mmHg of suction. However, rundown did occur so that increasing levels of suction were required to activate the channels over the 5 to 10 minutes during which data was acquired.

The open probability (P_{O}) was time and voltage dependent, displaying a fast adaptation (within 100 ms at hyperpolarized potentials) similar to that reported for Xenopus oocytes (Hamill et al., 1992, Proc. atl. Acad. Sci. USA, 89:7462-7466). The time dependence of P_{O} can be described by an initial phasic period followed by a tonic period as defined in (Bowman et al., 1996, Brain Res., 584:272-286). Both the duration of the phasic period and P_{O} during the tonic period showed a steep voltage dependence, decreasing as the potential becomes more negative (FIG. 3A, see average currents). Out of 16 cell-attached patches analyzed, 12 displayed adaptation at hyperpolarized potentials. In addition to adaptation, multiple voltage-dependent substates are visible at −100 mV, compared to only one at depolarizing potentials.

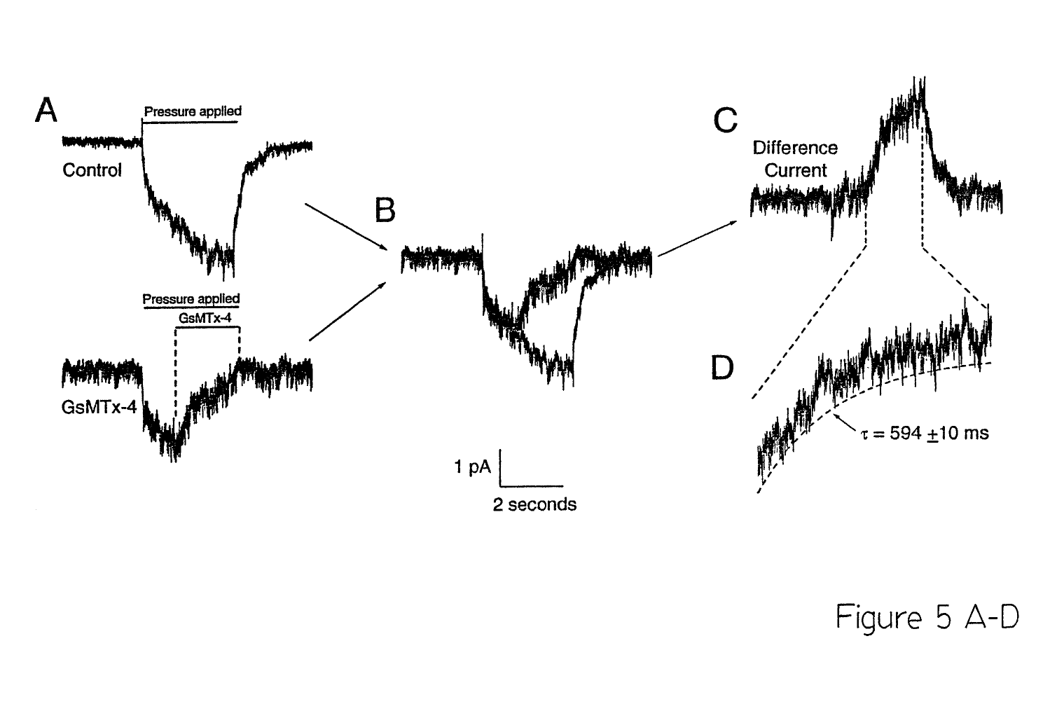

Although SACs had different adaptation properties in outside-out patches, channel activity was generally similar to that in cell attached patches. The SACs opened in response to both pressure and suction (FIG. 3B). With 140 mM KCl in both the pipette and bath the I-V profile (44 pS at −100 mV, and 21 pS at +100 mV, cytoplasmic side) was nearly identical to that observed for cell-attached patches (FIG. 3D). In this configuration the channels were initially activated by between 30-40 mmHg of pressure. The similarities between the conductance and pressure sensitivity in the two patch configurations suggest that these channel properties have not been significantly modified by outside-out patch formation. However, out of 12 outside-out patches, only 1 displayed the fast adaptation property observed in cell-attached patches. Instead, 2 showed no change in P_{O} with respect to time or voltage, while the remaining 9 patches exhibited a slow increase in current at both positive and negative voltages where the number of active channels increased during the 500 ms pressure step (see FIG. 3B 100 mV, and FIG. 5A average control current). The rate of increase was greater for pressure steps at positive voltages due to an increase in P_{O} at positive potentials. The single channel conductance and inward rectification observed here were similar to the properties reported for the family of nonselective cation SACs (for review see:Yang et al., 1993, In:Non-selective ion channels. D. Siemen and J. Hescheler, Eds. Springer Verlag, Heidelberg. pp79-92). These data indicate that outside-out patches can be used for the study of modulation of SAC function.

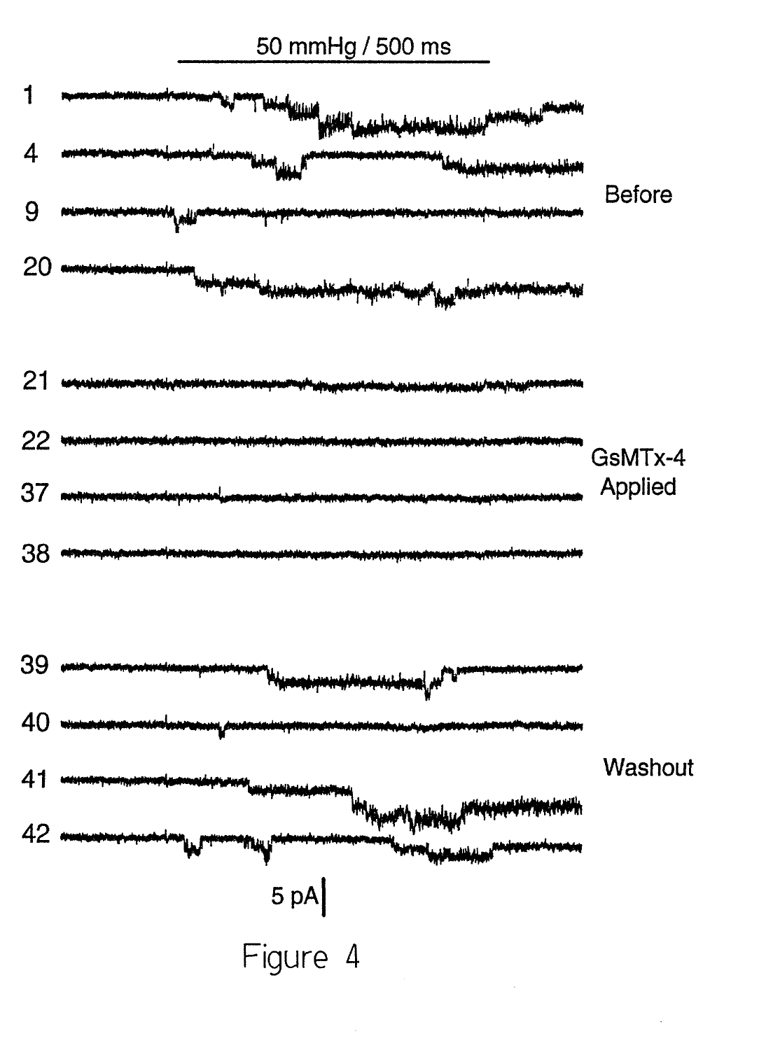

The outside out patches from adult astrocytes as described above were used for testing the HPLC fractions obtained in Example 1. The HPLC fractions were lyophilized, redissolved at a 1:1000 dilution and perfused onto outside out patches. Fraction 9 from FIG. 1A completely blocked SACs. The fraction containing the single peak in FIG. 1D was then tested for activity against SACs. The results are shown in FIG. 4. With this fraction, the block was complete, and occurred rapidly upon superfusion of the patch as shown by representative current traces in FIG. 4. The patch was held at −50 mV, and the pressure pulse is shown above the records. The entire experiment is comprised of 60 pressure steps: steps 1-20 occur before GsMTx-4 application; steps 21-38 while GsMTx-4 is being perfused; steps 39-60 occur during washout. Each 500 ms pressure step was separated by 1.5 seconds at 0 pressure. Four representative records from each stage of the experiment are displayed. Other peptides have been isolated from the spider venom which are active against SACs (such as, GsMTx-1, described in U.S. Pat. No. 5,756,663), however GsMTx-4 showed the most consistent and potent activity.

The association rate of the toxin was determined by applying GsMTx-4 to an outside-out patch while the channels were activated by stretch (FIG. 5). Average SAC currents were calculated from 3-second pressure steps indicated by the bars above the traces (A). The control trace was generated from 37 pressure steps applied to 7 different patches held at −50 mV, with pressure levels ranging from 35-70 mmHg. The current increased exponentially over the 3-second pressure application. The GsMTx-4 response was produced by applying 5 μM toxin one second after the onset to the pressure step indicated by GsMTx-4 bar. The GsMTx-4 current record was averaged from 29 pressure steps to 6 different patches held at −50 mV, with the steps ranging between 38-80 mmHg. Currents were nearly identical over the first second of the average current records as shown when the two are superimposed in (B). Subtracting the control current trace from the GsMTx-4 trace produced the difference current in (C). The current trace during GsMTx-4 application was fitted with a single exponential yielding a time constant of 594±10 ms (D). The fit is shown displaced from the data for clarity.

In the absence of GsMTx-4, channel activity increased over time at constant pressure (compare FIG. 3B with FIG. 5A). When 5 μM toxin was perfused onto the patch one second after the initiation of the pressure step, the current decayed exponentially (FIG. 5A, GsMTx-4). When the control and GsMTx-4 average current records are superimposed, the first second before GsMTx-4 application shows that the rate and amount of current increase are nearly identical (FIG. 5B). The difference current was calculated (FIG. 5C) and the period during which 5 μM GsMTx-4 was applied was fitted with a single exponential (FIG. 5D) yielding a time constant of 594±10 ms. Assuming a 1:1 binding, this gives an association constant, k_{A}, of 3.4×10⁵ M⁻¹ s⁻¹.

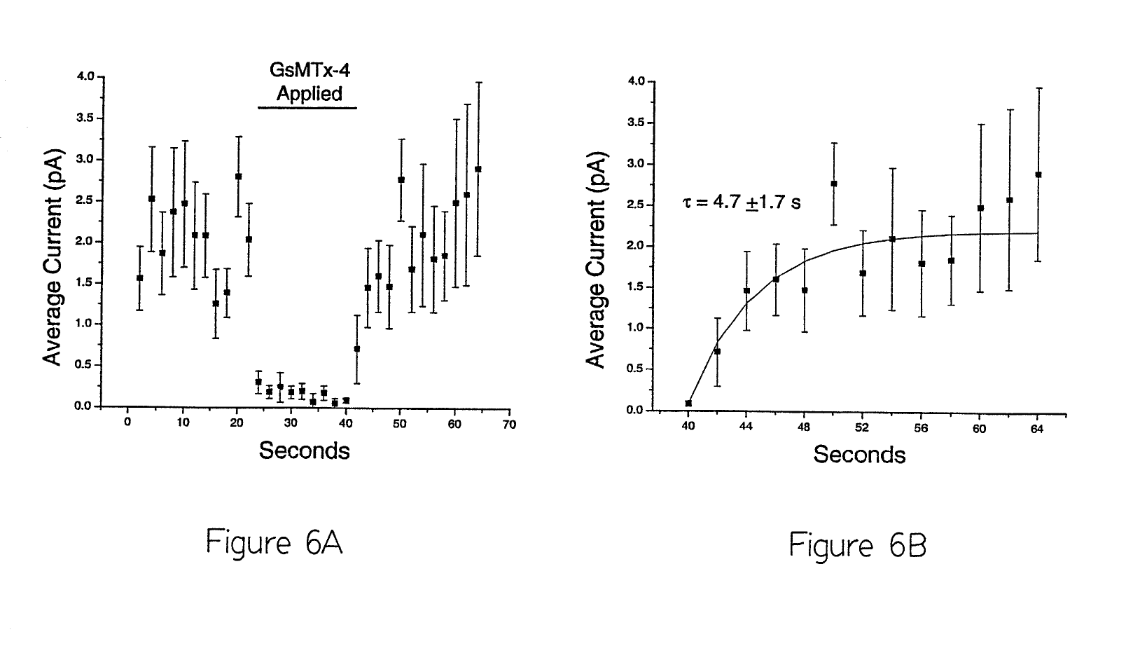

To determine the dissociation rate, the average patch current due to the opening of SACs was monitored before, during and after GsMTx-4 application from 7 different patches (FIGS. 6A and 6B). SAC currents were activated by 500 ms pressure steps at 2 second intervals in outside-out patches held at −50 mV. FIG. 6A represents the average current (± standard error) from 7 different patches. The recovery kinetics were fitted to a single exponential with a time constant of 4.7±1.7 seconds (FIG. 6B). From this dissociation constant (k_{d}=0.21 s^{31 1}) and the association constant determined above (kₐ=3.3×10⁵ M⁻¹ s⁻¹), the calculated equilibrium constant, K_{D}=k_{d}/kₐ=631 ±240 nM (standard error calculated from the first order approximation using the errors of kₐ and k_{d}). Using the ratio of rate constants to evaluate K_{D} minimizes errors caused by rundown. The mean SAC current was 2.04 ±0.14 pA (SE) over 11 pressure steps prior to GsMTx-4 application (FIG. 6A). When GsMTx-4 was applied, the average current dropped to 0.17±0.02 pA. The average current over the last 8 pressure steps (10 seconds after GsMTx-4 washout) returned to the initial current level of 2.28±0.17 pA. For a single binding site, Michealis-Menton knetics predicts the ratio of the blocked to the unblocked current is I/I₀=1 (1+K_{d}/S), where S is the substrate (peptide) concentration and K_{d} is the equilibrium dissociation constant. Using the data from FIG. 6, I/I₀=0.083, which gives a binding constant Kd 415 nM, consistent with the value calculated from the ratio of association and dissociation rates. There is no significant difference between whole-cell currents in isotonic saline and currents measured between 30 and 120 s after perfusion with 5 μM GsMTx-4. In contrast to GsMTx-4, CsCl produces a significant decrease in current at hyperpolarized potentials.

### EXAMPLE 3

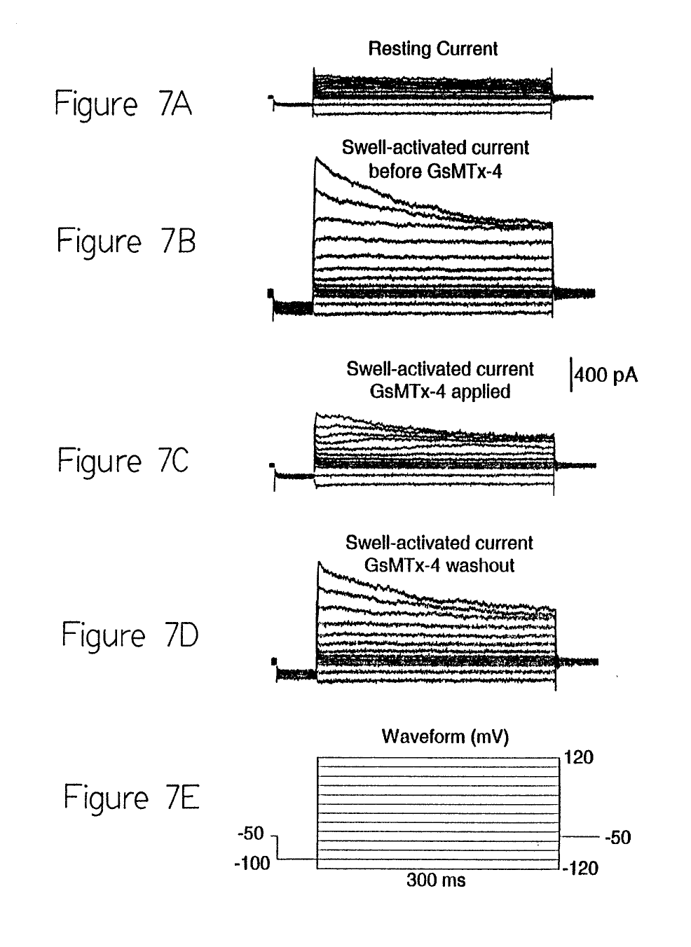

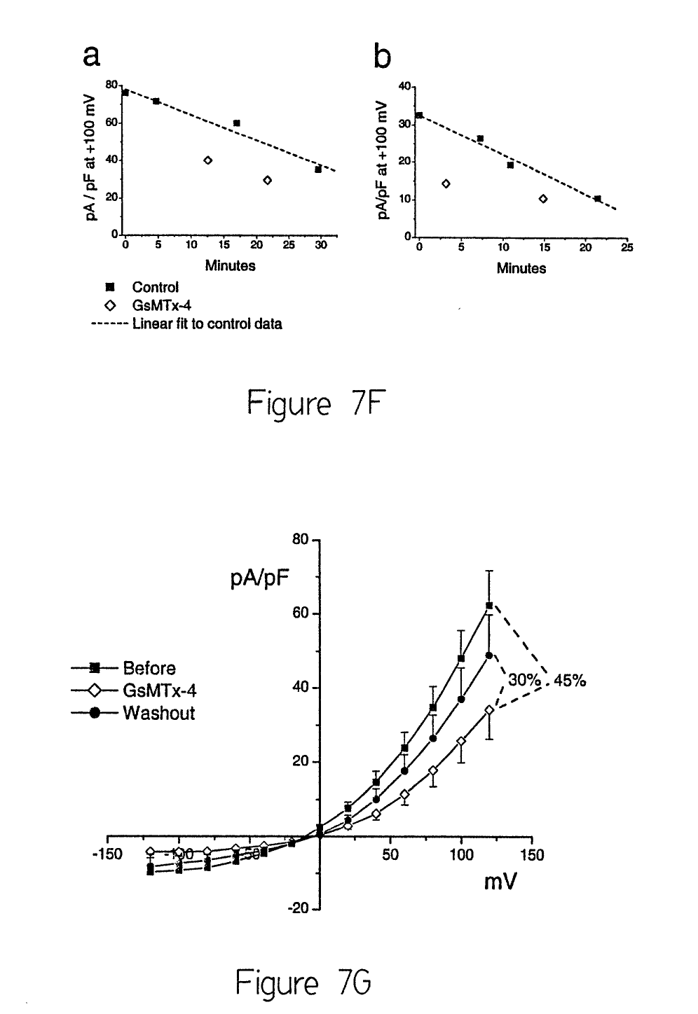

This example demonstrates the effect of GsMTx-4 on whole-cell swelling activated currents. It is considered that part of the swelling activated currents are attributable to SACs. To illustrate this embodiment, adult rat astrocytes were cultured as described above. Whole cell current was measured by the Nystatin perforated patch. Bath saline was the same as in Example 2. Pipette saline consisted of: KCl 80 mM, K₂SO₄ 30 mM, NaCl 10 mM, MgSO₄ 3 mM, CaCl₂ 0.13 mM, EGTA 0.23 mM and Hepes 10 mM, pH 7.3. Nystatin was dissolved in pipette saline to a final concentration of 200 mg/ml. After patch formation, access resistance was allowed to drop to ^{˜}15 MW (uncompensated), after which the series resistance compensation was set at ^{˜}65%, and prediction was set to ^{˜}75%. Whole cell capacitance measurements ranged from ^{˜}25-50 pF. Whole cell currents were monitored by either a voltage step protocol shown in FIG. 7, or by 600 ms voltage ramps. During hypotonic swelling the cell was perfused initially with isotonic saline (bath saline with 160 mM mannitol replacing 80 mM NaCl) before switching to hypotonic saline (isotonic saline minus 140 mM mannitol). The BPS-8 perfusion system was used to rapidly (<200 ms) change the bathing solution. Peak currents were measured at 3-5 ms into voltage steps.

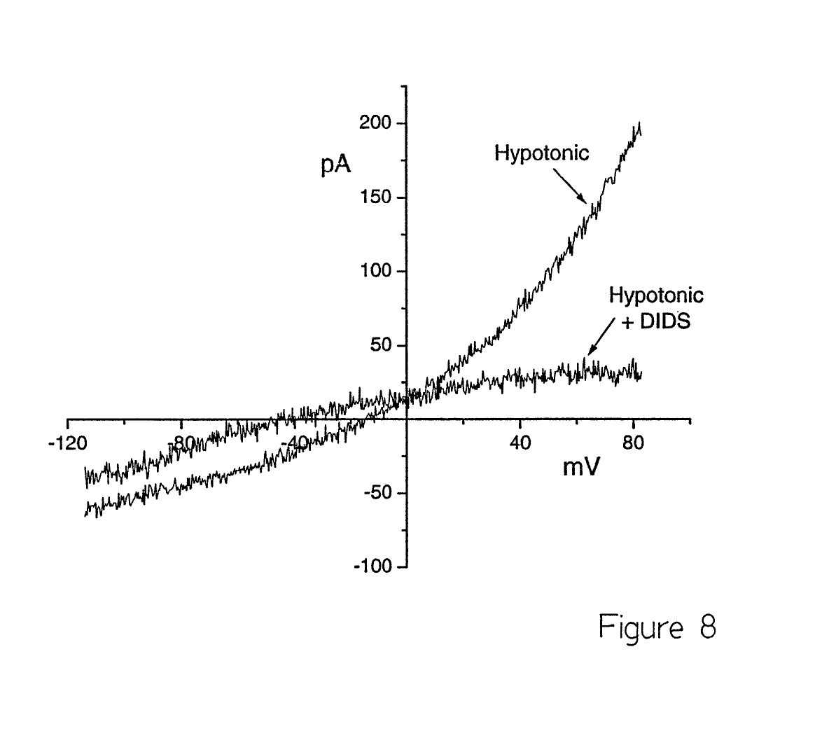

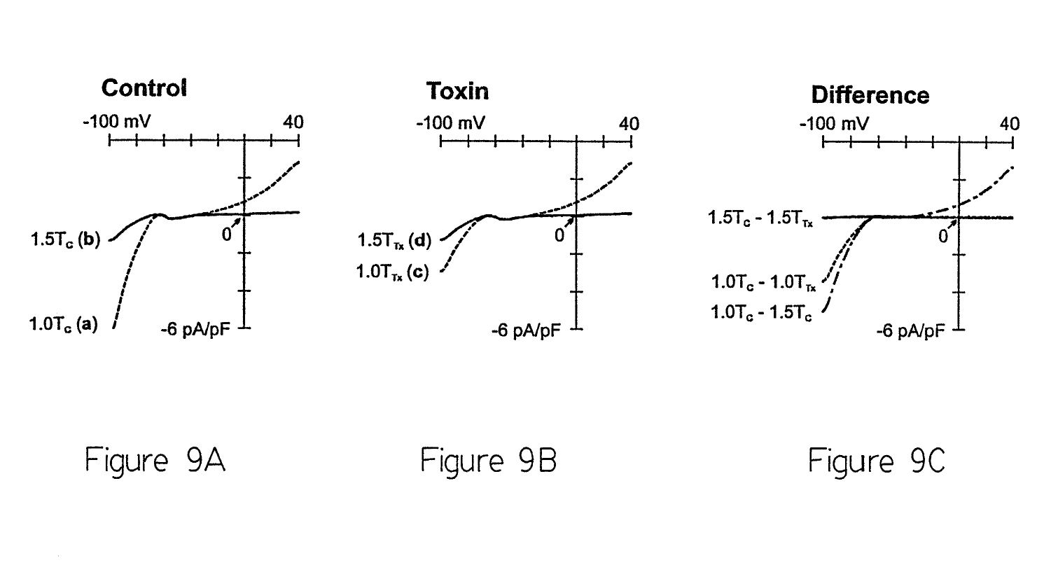

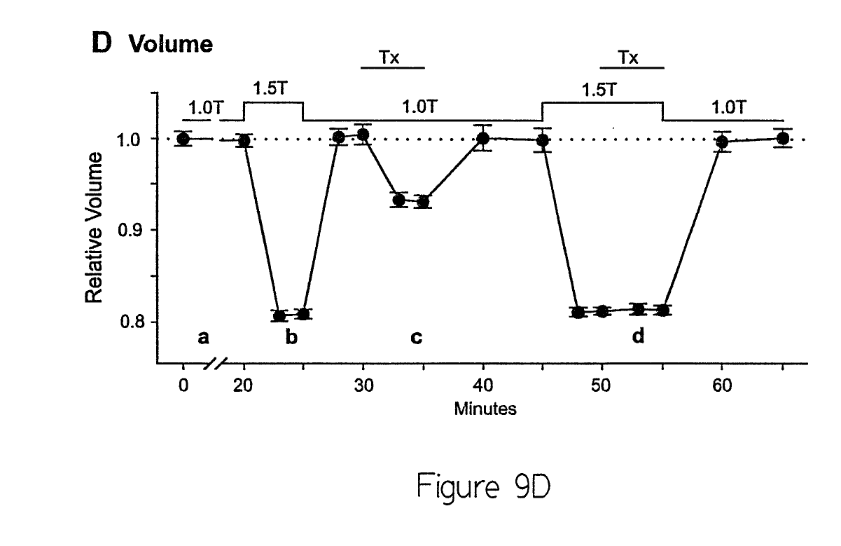

As shown in FIGS. 7A-G, the peptide GsMTx-4 reduces whole cell swelling activated currents. After 30 seconds exposure to hypotonic conditions, adult astrocytes display a similar large conductance increase that slowly inactivates at large depolarizing voltages as shown by perforated patch whole cell current recordings (compare FIG. 7A resting current to FIG. 9B swelling-activated current). During hypotonic exposure, cells were held at −50 mV prior to I-V test voltage steps to reduce the influence of voltage gated Ca²⁺ channels on Ca²⁺ influx. The swelling activated current has a large anionic component since 50 μM DIDS produced a significant reduction in current (especially at depolarized potentials) and an average −33 mV (n=6) shift in the reversal potential (FIG. 8). A residual current with a reversal potential shifted toward E_{K} remained. Applying 5 μM GsMTx-4 while hypotonically swelling the cell significantly reduced the peak current response by about 75% at 30 seconds after hypotonic exposure (FIG. 7C). After washout of GsMTx-4, the swelling currents partially recovered. A hypotonic stimulus produced larger swelling-activated currents, although not of the same magnitude as the original control stimulus (FIG. 7D). This reduced response after washout is not due to lingering toxin effects, since >3 min of washout separated successive hypotonic stimuli. Instead, it was observed that the response to successive hypotonic exposures slowly decreased over time (FIG. 7Fa and b). Representative peak current responses from two different cells displayed a roughly linear decrease in swelling-activated current (FIG. 7F). GsMTx-4 always reduced the swelling-activated current from the control response (FIG. 7F, ⋄). However, in light of the slowly degrading hypotonic response, it was necessary to estimate the amount of GsMTx-4 block by subtracting it from the mean “before” and “after” control hypotonic stimuli. The I-V profiles for the swelling-activated difference currents in FIG. 7G show a clear difference between the before (▪) and after (●) control responses. The percent block produced by GsMTx-4 (⋄) relative to each of the control curves is shown to the right. The estimated reduction in swelling-activated current produced by 5 μM GsMTx-4 was similar at both hyperpolarizing and depolarizing potentials (^{˜}48% at −100 mV and ^{˜}38% at+100 mV). Furthermore, unlike DIDS which produced a large (−33 mV) shift in reversal potential due to the specific loss of anionic current, GsMTx-4 produces almost no change in reversal potential (+2 mV, statistically indistinguishable from 0 mV). These data demonstrate that the peptide GsMTx-4 blocks SACs in swelling activated currents.

### EXAMPLE 4

This example demonstrates that the peptide GsMTx-4 affects stretch/swell induced currents in a model for congestive heart failure. Ventricular myocytes were freshly isolated from New Zealand white rabbits with aortic regurgitation induced congestive heart failure using a collagenase-pronase dispersion method (Clemo et al., 1997, supra). Cells were stored in a modified Kraft-Bruhe solution (KOH 132 mM, glutamic acid 120 mM, KCl 2.5 mM, KH₂PO₄ 10 mM, MgSO₄ 1.8 mM, K₂EGTA 0.5 MM, glucose 11 mM, taurine 10 mM, Hepes 10 mM, pH 7.2). Myocytes were used within 6 h of harvesting and only quiescent cells with no evidence of membrane blebbing were selected for study.

Swelling activated currents were measured in myocytes according to the method of Clemo & Baumgarten (1997). Briefly, electrodes were pulled from glass capillaries to give a final tip diameter of 3-4 mm and a resistance of 0.5-1 MW when filled with the standard electrode filling solution (K aspartate 120 mM, KCl 10 mM, NaCl 10 mM, MgSO₄ 3 mM, Hepes 10 mM, pH 7.1). Whole cell currents were recorded using an Axoclamp 200 A. Pulse and ramp protocols, voltage clamp data acquisition and off-line data analysis were controlled with software written in ASYST. Both step and ramp voltage clamp protocols were applied with a holding potential of −80 mV. Currents were digitized at 1 KHz and low-pass filtered at 200 Hz. Whole cell currents were recorded using the amphotericin perforated patch technique. Solution changes were performed by bath perfusion that was completed within 10 s. The standard bath solution contained (NaCl 65 mM, KCl 5 mM, CaSO₄ 2.5 mM, MgSO₄ 0.5 MM, glucose 10 mM and Hepes 10 mM pH 7.2, and 130 mM (1 T) or 283 mM (1.5 T) mannitol to control the osmolarity. Isotonic osmolarity was taken as 296 mosm (1 T) and 444 mosm for hypertonic solution (1.5 T).

The myocytes were exposed to 1.0 T and 1.5 T solution in the absence (1.0 T_{C}, 1.5 T_{C}) and presence (1.0 T_{TX}, 1.5 T_{TX}) of 0.4 μM GsMTx-4. As shown in FIG. 9, at 0.4 mM, GsMTx-4 produced a significant reduction of the inward I_{Cir,swell}, but had no effect on the outward I_{Cl,swell} (FIG. 9Bc). However, whole cell current was unaffected by GsMTx-4 when swelling-activated currents were inactivated by 1.5 T hypertonic saline (FIG. 9Bd). The difference currents in FIG. 9C show that GsMTx-4 blocked only inward swelling-activated current (compare: FIG. 9C, 1.0 T_{C}-1.5 T_{C} (total I_{Cir,swell}) to 1.0 T_{C}-1.0 T_{TX} (toxin sensitive I_{Cir,swell})). The remaining inward current is largely I_{Cl,swell}. The toxin produced no further reduction in the presence of hypertonic saline when swelling activated current is turned off (FIG. 9C, 1.5 T_{C}-1.5 T_{TX}).

Myocyte volume was determined by visualization with an inverted Nikon Diaphot microscope equipped with Hoffman modulation optics and a high resolution TV camera coupled to a video frame-grabber. Images were captured on-line each time a ramp or step voltage clamp protocol was performed using a program written in C and assembler and linked to ASYST voltage-clamp software. A combination of commercial (MOCHA; SPSS) and custom (ASYST) programs were used to determine cell width, length, and area of the image. The results of volume changes are shown in FIG. 9D. GsMTx-4 produced a cell volume reduction that is ^{˜}40% of that produced by 1.5 T hypotonic saline. These data indicate that GsMTx-4 blocks stretch activated channels in swelling activated currents.

### EXAMPLE 5

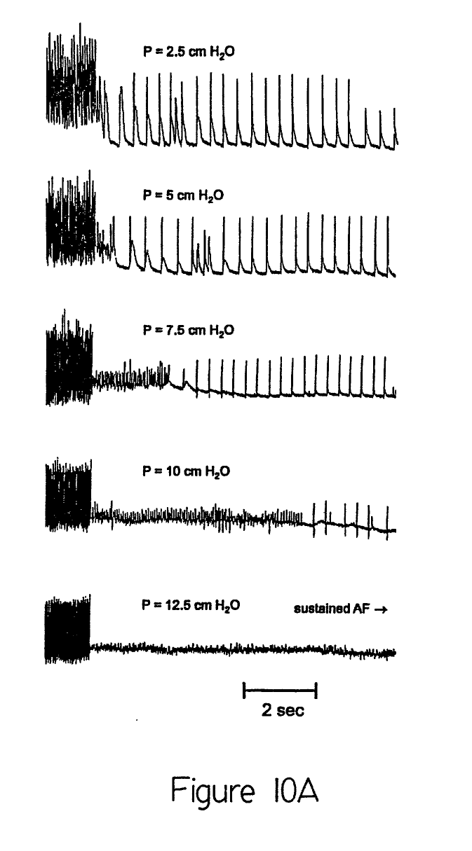

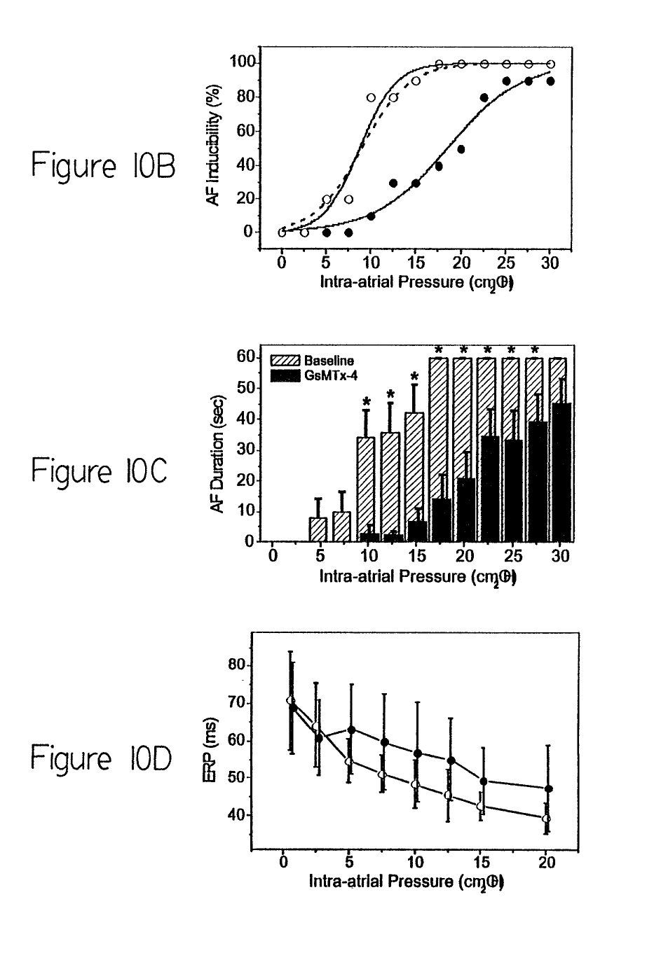

This example demonstrates that the peptide of the present invention can be used against cardiac fibrillation. To illustrate this embodiment, fibrillation was initiated in perfused rabbit hearts with a burst of high-frequency stimulation FIG. 10A. Stretching the atrium chamber increased the incidence and duration of fibrillation (FIG. 10A-C). At pressures above 12 cm H₂O, the fibrillation became sustained and the probability of sustained fibrillation (for longer than 60 s) approached unity. The probability of inducing fibrillation was increased by stimulating the heart with a short burst of high-frequency pacing before each measurement (arrow in FIG. 10b). Perfusion with 170 nM GsMtx-4 suppressed both the incidence (FIG. 10c) and duration (FIG. 10d) of fibrillation in all hearts (n=10). At pressures below 17.5 cm H₂O, sustained fibrillation was completely inhibited in all preparations (data not shown). GsMTX-4 did not block stretch-induced shortening of the refractory period.

The data presented in these Examples indicates that the peptide of the present invention blocks SACs in rat astrocytes, rabbit cardiac myocytes, and whole rabbit hearts in a stretch-dependant manner. Since stretch sensitivity is not unique to any particular chamber of the heart, GsMTx-4 can be used similarly on all chambers. This peptide should therefore be useful in elucidating the function of SACs in a variety of systems under physiologically normal and stressed conditions, and for blocking SACs associated with pathological conditions such as cardiac arrhythmias.

The foregoing description of the specific embodiments is for the purpose of illustration and is not to be construed as restrictive. From the teachings of the present invention, those skilled in the art will recognize that various modifications and changes may be made without departing from the spirit of the invention.

We claim:

1. An isolated peptide having the amino acid sequence of SEQ ID No. 1.

2. A pharmaceutical composition comprising the peptide of claim 1 in a pharmaceutically acceptable carrier or diluent.

3. A method of reducing cardiac arrhythmia comprising administering the peptide of claim 1 in a pharmaceutically acceptable carrier to a patient in need of treatment.

4. A method of blocking stretch-activated channels in a cell comprising the step of contacting the cell with a sufficient amount of the peptide of claim 1.

5. The method of claim 4, wherein the cell is a myocardial cell.

6. The method of claim 4, wherein the cell is an astrocyte.


SEQUENCE LISTING (1 sequence)

SEQ ID NO: 1 - protein, 35 aa, Grammastola spatulata
GCLEFWWKCNPNDDKCCRPKLKCSKLFKLCNFSSG